(19) United States
(12) Patent Application Publication
(10) Pub. No.: US 2019/0336494 A1
(43) Pub. Date: Nov. 7, 2019

(54) DEUTERATED MORPHINAN COMPOUNDS FOR TREATING AGITATION

(71) Applicant: Concert Pharmaceuticals, Inc., Lexington, MA (US)

(72) Inventors: Roger D. Tung, Lexington, MA (US); Philip B. Graham, Boston, MA (US)

(21) Appl. No.: 16/518,776

(22) Filed: Jul. 22, 2019

Related U.S. Application Data
(62) Division of application No. 15/748,886, filed on Jan. 30, 2018, now abandoned.
(60) Provisional application No. 62/199,004, filed on Jul. 30, 2015.

(51) Int. Cl. A61K 31/485; A61P 25/16; A61P 25/18; A61P 25/28; A61P 25/24

(52) U.S. Cl. CPC A61K 31/485; A61P 25/16; A61K 31/49; A61P 25/28; A61P 25/24; A61P 25/18

(57) ABSTRACT

This invention relates to methods of treating agitation comprising administering a morphinan compound or a pharmaceutically acceptable salt thereof. This invention also provides the use in methods of treating agitation and related disorders with such a morphinan compound in combination with quinidine, or pharmaceutically acceptable salt of either or both thereof.

### CROSS-REFERENCE TO RELATED APPLICATIONS

This application is a divisional application of U.S. National Stage application 15/748,886, filed Jan. 30, 2018 which claims the benefit of International Application No. PCT/US2016/044885, having an International filing date of Jul. 29, 2016, which claims the benefit of U.S. Provisional Ser. No. 62/199,004 filed Jul. 30, 2015. The disclosure of the prior applications are considered part of (and are incorporated by reference in) the disclosure of this application.

### TECHNICAL FIELD

This invention relates to methods of treating agitation comprising administering a deuterated morphinan compound or a pharmaceutically acceptable salt thereof. This invention also provides the use of such a deuterated morphinan compound in combination with quinidine, or a pharmaceutically acceptable salt of either or both thereof, in methods of treating agitation and related disorders.

### BACKGROUND

Dextromethorphan, also known by its chemical name (+)-3-methoxy-17-methyl-(9α, 13α,14α)-morphinan, is currently one of the most widely used antitussives.

In addition to the physiological activity noted above, dextromethorphan is also an agonist of the σ2 receptor, an N-methyl-D-aspartate (NMDA) antagonist, and an α3β4 nicotinic receptor antagonist. Dextromethorphan inhibits neurotransmitters, such as glutamate, from activating receptors in the brain. Uptake of dopamine and serotonin are also inhibited.

Dextromethorphan is approved for use in over the counter cough suppressant products. It is currently in clinical trials for treating subjects with voice spasms, and for treating Rett Syndrome (http://www. clinicaltrials.gov). Dextromethorphan is also being studied in combination with other drugs in a clinical trial characterizing pain processing mechanisms in subjects with irritable bowel syndrome (http://www.clinicaltrials.gov/).

In addition, a combination of dextromethorphan hydrobromide and quinidine sulfate is currently in clinical trials for treating diabetic neuropathic pain, central neuropathic pain in multiple sclerosis, agitation in Alzheimer's patients, autism, and major depressive disorder (http://www.clinicaltrials.gov). This drug combination, also known as NUEDEXTA®, is approved for treating Involuntary Emotional Expression Disorder (IEED), also known as pseudobulbar affect.

Dextromethorphan is metabolized in the liver. Degradation begins with O— and N— demethylation to form primary metabolites dextrorphan and 3-methoxy-morphinan, both of which are further N— and O— demethylated respectively to 3-hydroxy-morphinan. These three metabolites are believed to be therapeutically active. A major metabolic catalyst is the cytochrome P450 enzyme 2D6 (CYP2D6), which is responsible for the O-demethylation reactions of dextromethorphan and 3-methoxymorphinan. N-demethylation of dextromethorphan and dextrorphan are catalyzed by enzymes in the related CYP3A family. Conjugates of dextrorphan and 3-hydroxymorphinan can be detected in human plasma and urine within hours of its ingestion.

Dimemorfan, an analog of dextromethorphan, also known by its chemical name as (+)-(9α, 13α, 14α)-3,17-dimethylmorphinan, is a non-narcotic antitussive. The antitussive activity of dimemorfan is believed to result from direct action on the cough center in the medulla (Ida, H., Clin Ther., 1997, Mar-Apr;19(2): 215-31).

In addition to its antitussive properties, dimemorfan has been shown to have anticonvulsant and neuroprotective effects possibly arising from N-methyl-D-aspartate (NMDA) antagonism of dextromethorphan (DM) and/or high-affinity DM σ receptors (Chou, Y-C. et al., Brain Res., 1999, Mar 13;821(2): 516-9). Activation at the σ-1 receptor has been found to provide anticonvulsant action in rats and mice, like DM, but without the behaviorial side effects produced by DM and its metabolite, dextrorphan (Shin, E. J. et al., Br J Pharmacol., 2005, Apr;144(7): 908-18 and Shin, E. J. et al., Behavioural Brain Research, 2004, 151: 267-276).

Metabolism of dimemorfan in humans is known to proceed through cytochrome P450 catalyzed N-demethylation as well as 3-methyl oxidation. Greater than 98% of a dose of dimemorfan is metabolized in healthy human males and none of the metabolites have been shown to have antitussive effects (Chou Y-C., et al., Life Sci., 2005, Jul. 1;77(7): 735-45 and Chou Y-C., et al., J Pharm Sci., 2009, Jul. 1-15).

### SUMMARY

Provided herein is a method of treating agitation comprising administering to a subject in need thereof, an effective amount of a compound of Formula I:

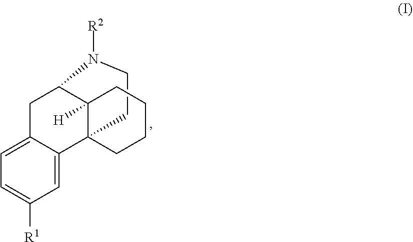

or a pharmaceutically acceptable salt thereof, wherein:

R¹ is selected from —CH₃, —CH₂D, —CHD₂, and —CD₃; and

R² is selected from —CH₃, —CH₂D, —CHD₂, and —CD₃;

provided that either R¹ or R² comprises at least one deuterium atom;

and a pharmaceutically acceptable carrier.

In some embodiments, R¹ is —CH₃ or —CD₃ and R² is —CH₃ or —CD₃.

In some embodiments, a Formula I compound is selected from any one of the compounds in Table 1 set forth below:

TABLE 1
Compounds of Formula I

| Compound No. | R¹ | R² |
|---|---|---|
| 100 | —CD₃ | —CD₃ |
| 101 | —CH₃ | —CD₃ |
| 102 | —CD₃ | —CH₃, |

or a pharmaceutically acceptable salt thereof, wherein any atom not designated as deuterium is present at its natural isotopic abundance.

In some embodiments, for the compound of Formula I, any atom not designated as deuterium is present at its natural isotopic abundance.

In some embodiments, the method comprises administering to the subject an amount of quinidine, or a pharmaceutically acceptable salt thereof, wherein the amount of quinidine, or a pharmaceutically acceptable salt thereof, is in the range of 1 mg/day to 40 mg/day.

In some embodiments, the method comprises administering an amount of compound of Formula I, or a pharmaceutically acceptable salt thereof, in the range of 5 mg/day to 250 mg/day.

In some embodiments, the method comprises administering an amount of compound of Formula I, or a pharmaceutically acceptable salt thereof, in the range of 5 mg/day to 250 mg/day and the amount of quinidine, or a pharmaceutically acceptable salt thereof, is in the range of 1 mg/day to 20 mg/day.

In some embodiments, the agitation is associated with a disorder selected from the group consisting of Alzheimer's disease, a degenerative neurological disorder, a mood disorder, substance abuse withdrawal, selective serotonin reuptake inhibitor (SSRI) withdrawal, withdrawal from benzodiazepines, withdrawal from drugs useful for the treatment of attention deficit disorder (ADD) and attention deficit hyperactive disorder (ADHD), traumatic brain injury, terminal illness, post-operative agitation, post-anesthetic agitation, Reye's syndrome and a pediatric disorder.

In some embodiments, the agitation is associated with a degenerative neurological disorder. In one aspect of these embodiments, the degenerative neurological disorder is Parkinson's disease. In another aspect of these embodiments, the degenerative neurological disorder is Huntington's disease.

In some embodiments, the agitation is associated with a mood disorder. In one aspect of these embodiments, the mood disorder is depression, dysthymia, schizophrenia or bipolar disorder. In one specific aspect of these embodiments, the mood disorder is depression. In one specific aspect of these embodiments, the mood disorder is dysthymia. In one specific aspect of these embodiments, the mood disorder is schizophrenia. In one specific aspect of these embodiments, the mood disorder is bipolar disorder.

In some embodiments, the agitation is associated with SSRI withdrawal. In one aspect of these embodiments, the SSRI is selected from fluoxetine, fluvoxamine, citalopram, escitalopram, paroxetine and sertraline.

In some embodiments, the agitation is associated with withdrawal from drugs useful for the treatment of ADD and ADHD. In one aspect of these embodiments, the drug useful for the treatment of ADD and ADHD is selected from methamphetamine hydrochloride, methylphenidate hydrochloride, dextroamphetamine sulfate, mixed amphetamine salts, pemoline, dexmethylphenidate hydrochloride, and lisdexamfetamine mesilate.

In some embodiments, the agitation is associated with a pediatric disorder. In one aspect of these embodiments, the pediatric disorder is depression, attention deficit disorder, oppositional defiant disorder, or separation anxiety disorder.

In some embodiments, the agitation is associated with Alzheimer's disease.

In some embodiments, the agitation is associated with traumatic brain injury.

Also provided herein is a method of treating a disease or disorder selected from the group consisting of diabetes, epilepsy, and depression, comprising administering to a subject in need thereof an effective amount of a compound of Formula I:

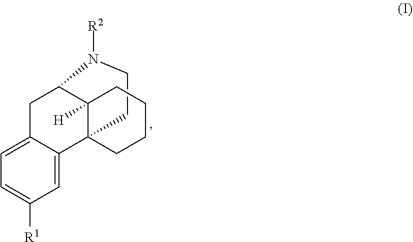

or a pharmaceutically acceptable salt thereof, wherein:

R¹ is selected from —CH₃, —CH₂D, —CHD₂, and —CD₃; and

R² is selected from —CH₃, —CH₂D, —CHD₂, and —CD₃;

provided that either R¹ or R² comprises at least one deuterium atom;

together with quinidine or a pharmaceutically acceptable salt thereof, and optionally a pharmaceutically acceptable carrier.

In some embodiments, R¹ is —CH₃ or —CD₃ and R² is —CH₃ or —CD₃.

In some embodiments, the Formula I compound is selected from any one of the compounds in Table 1 set forth below:

TABLE 1
Compounds of Formula I

| Compound No. | R¹ | R² |
|---|---|---|
| 100 | —CD₃ | —CD₃ |
| 101 | —CH₃ | —CD₃ |
| 102 | —CD₃ | —CH₃, |

or a pharmaceutically acceptable salt thereof, wherein any atom not designated as deuterium is present at its natural isotopic abundance.

In some embodiments, for the compound of Formula I, any atom not designated as deuterium is present at its natural isotopic abundance.

In some embodiments, the amount of the compound of Formula I, or a pharmaceutically acceptable salt thereof, is in the range of 5 mg/day to 500 mg/day, and the amount of quinidine, or a pharmaceutically acceptable salt thereof, is in the range of 1 mg/day to 40 mg/day. In some embodiments, the amount of the compound of Formula I, or a pharmaceutically acceptable salt thereof, is in the range of 5 mg/day to 250 mg/day and the amount of quinidine, or a pharmaceutically acceptable salt thereof, is in the range of 1 mg/day to 20 mg/day. In some embodiments, the amount of the compound of Formula I, or a pharmaceutically acceptable salt thereof, is in the range of 10 mg/day to 150 mg/day and the amount of quinidine, or a pharmaceutically acceptable salt thereof, is in the range of 1 mg/day to 20 mg/day.

### DETAILED DESCRIPTION

### Definitions

The terms “ameliorate” and “treat” are used interchangeably and include both therapeutic treatment and/or prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, or organ.

As used herein, the term “subject” includes humans and non-human mammals. Non-limiting examples of non-human mammals include mice, rats, guinea pigs, rabbits, dogs, cats, monkeys, apes, pigs, cows, sheep, horses, etc.

It will be recognized that some variation of natural isotopic abundance occurs in a synthesized compound depending upon the origin of chemical materials used in the synthesis. Thus, a preparation of dextromethorphan or dextromethorphan analogs will inherently contain small amounts of deuterated isotopologues. The concentration of naturally abundant stable hydrogen and carbon isotopes, notwithstanding this variation, is small and immaterial as compared to the degree of stable isotopic substitution of compounds of this invention. See, for instance, Wada E et al., Seikagaku 1994, 66:15; Gannes LZ et al., Comp Biochem Physiol Mol Integr Physiol 1998, 119:725.

In the compounds of this invention any atom not specifically designated as a particular isotope is meant to represent any stable isotope of that atom. Unless otherwise stated, when a position is designated specifically as “H” or “hydrogen”, the position is understood to have hydrogen at its natural abundance isotopic composition. Also unless otherwise stated, when a position is designated specifically as “D” or “deuterium”, the position is understood to have deuterium at an abundance that is at least 3340 times greater than the natural abundance of deuterium, which is 0.015% (i.e., the term “D” or “deuterium” indicates at least 50.1% incorporation of deuterium).

The term “isotopic enrichment factor” as used herein means the ratio between the isotopic abundance of D at a specified position in a compound of this invention and the naturally occurring abundance of that isotope. The natural abundance of deuterium is 0.015%.

In other embodiments, a compound of this invention has an isotopic enrichment factor for each deuterium present at a site designated as a potential site of deuteration on the compound of at least 3500 (52.5% deuterium incorporation), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

The term “isotopologue” refers to a species that has the same chemical structure and formula as a specific compound of this invention, with the exception of the positions of isotopic substitution and/or level of isotopic enrichment at one or more positions, e.g., H vs. D.

The term “compound,” as used herein, refers to a collection of molecules having an identical chemical structure, except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this invention will depend upon a number of factors including the isotopic purity of deuterated reagents used to make the compound and the efficiency of incorporation of deuterium in the various synthesis steps used to prepare the compound.

A salt of a compound of this invention is formed between an acid and a basic group of the compound, such as an amino functional group, or a base and an acidic group of the compound, such as a carboxyl functional group. According to another embodiment, the compound is a pharmaceutically acceptable acid addition salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any suitable salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-l-sulfonate, naphthalene-2- sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The term “stable compounds,” as used herein, refers to compounds which possess stability sufficient to allow for their manufacture and which maintain the integrity of the compound for a sufficient period of time to be useful for the purposes detailed herein (e.g., formulation into therapeutic products, intermediates for use in production of therapeutic compounds, isolatable or storable intermediate compounds, treating a disease or condition responsive to therapeutic agents).

“Stereoisomer” refers to both enantiomers and diastereomers. “D” refers to deuterium. “Tert”, “ ^{t}”, and “t” each refer to tertiary. “US” refers to the United States of America. “FDA” refers to Food and Drug Administration. “NDA” refers to New Drug Application. “rt” and “RT” refer to room temperature. “h” refers to hours. “DMF” refers to dimethylformamide. “T_{S}OH” refers to p-toluenesulfonic acid.

Throughout this specification, a variable may be referred to generally (e.g.,“each R”) or may be referred to specifically (e.g., R¹ or R²). Unless otherwise indicated, when a variable is referred to generally, it is meant to include all specific embodiments of that particular variable.

### Therapeutic Compounds

Described herein are methods useful in the treatment of agitation such as agitation associated with Alzheimer's disease or traumatic brain injury in a patient in need thereof. Also described herein are methods for treating a disease or disorder selected from the group consisting of diabetes, epilepsy, and depression. In certain embodiments, treatment comprises the administration of a compound of Formula I:

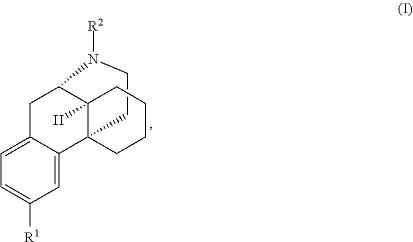

or a pharmaceutically acceptable salt thereof, wherein:

R¹ is selected from —CH₃, —CH₂D, —CHD₂, and —CD₃; and

R² is selected from —CH₃, —CH₂D, —CHD₂, and —CD₃;

provided that either R¹ or R² comprises at least one deuterium atom.

In some embodiments, treatment comprises the administration of a compound of Formula I, as described herein, and a pharmaceutically acceptable carrier.

In some embodiments, R¹ is —CH₃ or —CD₃ and R² is —CH₃ or —CD₃.

In some embodiments, R¹ is —CH₃ or —CD₃. In one aspect of these embodiments, R² is —CH₃. In one aspect of these embodiments, R² is —CD₃.

In some embodiments, R¹ is -CH3. In one aspect of these embodiments, R² is —CD₃.

In some embodiments, R¹ is -CD3. In one aspect of these embodiments, R² is —CH₃. In one aspect of these embodiments, R² is —CD₃.

In another set of embodiments, the compounds of Formula I are provided in isolated form, e.g., the compound is not in a cell or organism and the compound is separated from some or all of the components that typically accompany it in nature.

In some embodiments of the compounds of Formula I, any atom not designated as deuterium is present at its natural isotopic abundance.

In some embodiments, a Formula I compound is selected from any one of the compounds in Table 1 set forth below:

TABLE 1
Compounds of Formula I

| Compound No. | R¹ | R² |
|---|---|---|
| 100 | —CD₃ | —CD₃ |
| 101 | —CH₃ | —CD₃ |
| 102 | —CD₃ | —CH₃ |

or a pharmaceutically acceptable salt thereof, wherein any atom not designated as deuterium is present at its natural isotopic abundance.

In some embodiments, the compound of Formula I is selected from any one of:

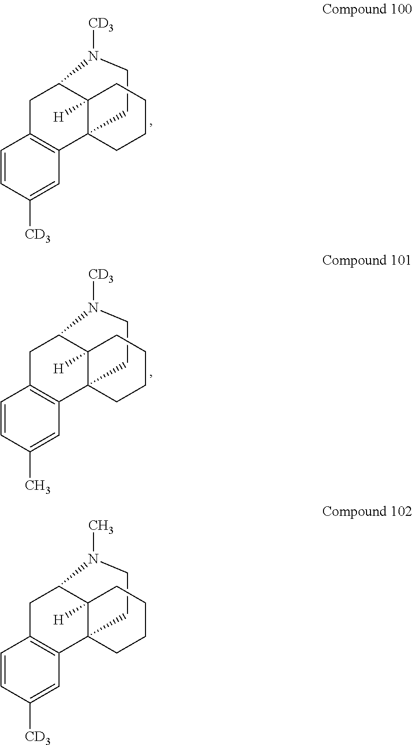 [SMILES: [2H]C([2H])([2H])c1ccc2c(c1)[C@]13CCCC[C@@H]1[C@H](C2)N(C([2H])([2H])[2H])CC3 ; [2H]C([2H])([2H])N1CC[C@@]23CCCC[C@@H]2[C@@H]1Cc1ccc(C)cc13 ; [2H]C([2H])([2H])c1ccc2c(c1)[C@]13CCCC[C@@H]1[C@H](C2)N(C)CC3]

or a pharmaceutically acceptable salt thereof.

In some embodiments, the method comprises administering an amount of compound of Formula I, or a pharmaceutically acceptable salt thereof, in the range of 1 mg/day to 1000 mg/day. In one aspect of these embodiments, the amount of compound of Formula I, or a pharmaceutically acceptable salt thereof, is in the range of 5 mg/day to 500 mg/day. In one aspect of these embodiments, the amount of compound of Formula I, or a pharmaceutically acceptable salt thereof, is in the range of 5 mg/day to 400 mg/day. In one aspect of these embodiments, the amount of compound of Formula I, or a pharmaceutically acceptable salt thereof, is in the range of 5 mg/day to 250 mg/day. In one aspect of these embodiments, the amount of compound of Formula I, or a pharmaceutically acceptable salt thereof, is in the range of 10 mg/day to 100 mg/day.

In some embodiments, treatment comprises administration of a combination of a compound of Formula I, as described herein, and quinidine, or a pharmaceutically acceptable salt of either or both thereof.

In some embodiments, treatment comprises the administration of a combination of a compound of Formula I, as described herein, quinidine, or a pharmaceutically acceptable salt of either or both thereof, and a pharmaceutically acceptable carrier.

In some embodiments, the method comprises administering to the subject an amount of quinidine, or a pharmaceutically acceptable salt thereof, wherein the amount of quinidine, or a pharmaceutically acceptable salt thereof, is in the range of 1 mg/day to 60 mg/day. In one aspect of these embodiments, the amount of quinidine is in the range of 1 mg/day to 40 mg/day. In one aspect of these embodiments, the amount of quinidine is in the range of 1 mg/day to 30 mg/day. In one aspect of these embodiments, the amount of quinidine is in the range of 1 mg/day to 20 mg/day. In one aspect of these embodiments, the amount of quinidine is in the range of 1 mg/day to 10 mg/day. In one aspect of these embodiments, the amount of quinidine is in the range of 1 mg/day to 5 mg/day.

In certain embodiments, the amount of quinidine is an amount effective to increase the plasma half-life or decrease the intrinsic clearance of the compound of Formula I in the subject by at least 50%, 100%, 200%, 300%, 400%, 500%, 600%, 700%, 800%, 900%, or 1000%, relative to the plasma half-life or the intrinsic clearance of the compound of Formula I in the subject in the absence of quinidine.

In some embodiments, the agitation is associated with a disorder selected from the group consisting of Alzheimer's disease, a degenerative neurological disorder, a mood disorder, substance abuse withdrawal, selective serotonin reuptake inhibitor (SSRI) withdrawal, withdrawal from benzodiazepines, withdrawal from drugs useful for the treatment of ADD and ADHD, traumatic brain injury, terminal illness, post-operative agitation, post-anesthetic agitation, Reye's syndrome and a pediatric disorder.

In some embodiments, the agitation is associated with a degenerative neurological disorder. In one aspect of these embodiments, the degenerative neurological disorder is Parkinson's disease. In another aspect of these embodiments, the degenerative neurological disorder is Huntington's disease.

In some embodiments, the agitation is associated with a mood disorder. In one aspect of these embodiments, the mood disorder is depression, dysthymia, schizophrenia or bipolar disorder. In one specific aspect of these embodiments, the mood disorder is depression. In one specific aspect of these embodiments, the mood disorder is dysthymia. In one specific aspect of these embodiments, the mood disorder is schizophrenia. In one specific aspect of these embodiments, the mood disorder is bipolar disorder.

In some embodiments, the agitation is associated with SSRI withdrawal. In one aspect of these embodiments, the SSRI is selected from fluoxetine, fluvoxamine, citalopram, escitalopram, paroxetine and sertraline.

In some embodiments, the agitation is associated with withdrawal from drugs useful for the treatment of ADD and ADHD. In one aspect of these embodiments, the drug useful for the treatment of ADD and ADHD is selected from methamphetamine hydrochloride, methylphenidate hydrochloride, dextroamphetamine sulfate, mixed amphetamine salts, pemoline, dexmethylphenidate hydrochloride, and lisdexamfetamine mesilate.

In some embodiments, the agitation is associated with a pediatric disorder. In one aspect of these embodiments, the pediatric disorder is depression, attention deficit disorder, oppositional defiant disorder, or separation anxiety disorder.

In some embodiments, the agitation is associated with Alzheimer's disease.

In some embodiments, the agitation is associated with traumatic brain injury.

In another set of embodiments, the compound of Formula I is purified, e.g., the compound of Formula I is present at a purity of at least 50.1% by weight (e.g., at least 52.5%, 55%, 60%, 65%, 70%, 75%, 80%, 85%, 90%, 95%, 97%, 98%, 98.5%, 99%, 99.5% or 99.9%) of the total amount of isotopologues of Formula I present, respectively. Thus, in some embodiments, a composition comprising a compound of Formula I can include a distribution of isotopologues of the compound, provided at least 50.1% of the isotopologues by weight are the recited compound.

In another set of embodiments, the compounds of Formula I are provided in isolated form, e.g., the compound is not in a cell or organism and the compound is separated from some or all of the components that typically accompany it in nature.

In some embodiments, any position in the compound of Formula I designated as having D has a minimum deuterium incorporation of at least 50.1% (e.g., at least 52.5%, at least 60%, at least 67.5%, at least 75%, at least 82.5%, at least 90%, at least 95%, at least 97%, at least 99%, or at least 99.5%) at the designated position(s) of the compound of Formula I.

In some embodiments, a compound of Formula I is “substantially free of” other isotopologues of the compound, e.g., less than 49.9%, less than 25%, less than 10%, less than 5%, less than 2%, less than 1%, or less than 0.5% of other isotopologues are present.

The synthesis of compounds of Formula I can be readily achieved by reference to the Exemplary Syntheses and Examples disclosed herein and in PCT application WO2010062690, and by use of procedures and intermediates analogous to those disclosed, for instance, in Schnider, O. & Grussner, A., Hely. Chim. Acta., 1951, 34: 2211; Grussner, A. & Schnider, O.; G B 713146 (1954); Toyo Pharma K. K., Japan JP 60089474 A (1983); Newman, A. H. et al., J. Med. Chem., 1992, 35: 4135. Such methods can be carried out by utilizing corresponding deuterated and, optionally, other isotope-containing reagents and/or intermediates to synthesize the compounds delineated herein, or by invoking standard synthetic protocols known in the art for introducing isotopic atoms to a chemical structure.

### Exemplary Syntheses

A convenient method for synthesizing compounds of Formula I wherein R¹ is —CH₃ or —CD₃ is depicted in Scheme 1.

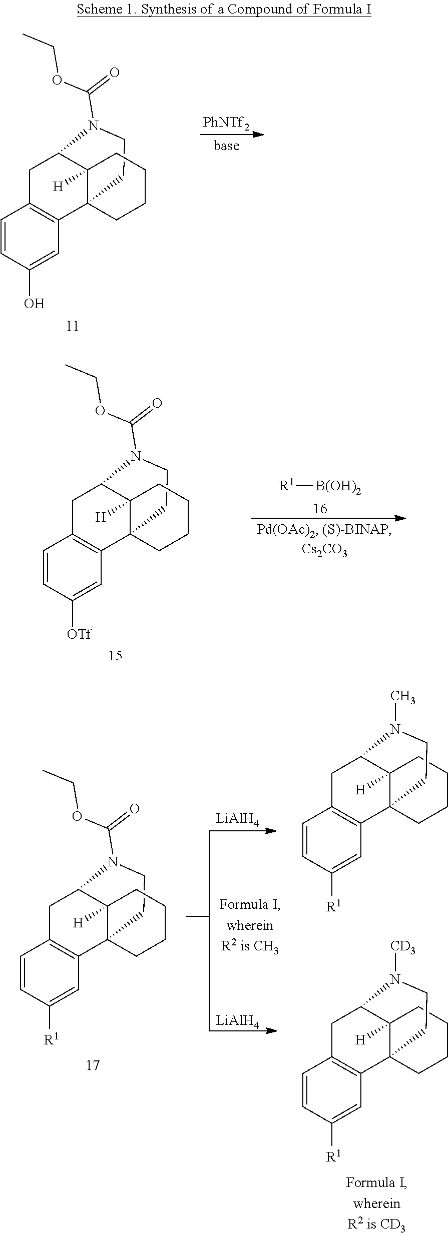

Treatment of 17-ethoxycarbonyl-3-hydroxy-morphinan (11) (see PCT application WO2010062690) with N-Phenyl-trifluoromethanesulfonimide according to the procedure described by Kim, C.-H. in US 2005/0256147 A1 affords the corresponding phenolic triflate (15). Palladium catalyzed cross-coupling of 15 with the appropriately deuterated methyl boronic acid (16) in a manner analogous to the procedure from the aforementioned patent gives the appropriately deuterated 17-ethoxycarbonyl-3-methyl-morphinans (17). Reduction of the carbamate of morphinan 17 with either lithium aluminum hydride or lithium aluminum deuteride in THF in a manner analogous to the procedure described by Newman, A. H. et al., Journal of Medicinal Chemistry 1992, 35: 4135-4142 affords the appropriately deuterated 3-methyl-17-methyl-morphinan or 3-methyl-17-trideuteromethyl-morphinan compounds of Formula I, respectively.

The alkylboronic acid reagent 16 used in Scheme 1 above is prepared as described in Scheme 2.

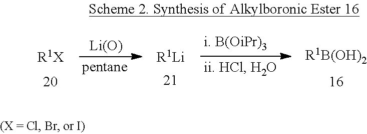

Treatment of appropriately deuterated R₁-halide (20) with elemental lithium in pentane in a manner analogous to the procedure described by Dawildowski, D. et al., in WO 2005/082911 A1 affords the corresponding R¹-lithium anion (21), which may be immediately treated with triisopropyl borate followed by hydrolysis with aqueous hydrogen chloride in a manner analogous to the procedure described by Brown, H. C. et al., Organometallics 1985, 4: 816-821 to afford the appropriately deuterated R¹-boronic acids (16).

The specific approaches and compounds shown above are not intended to be limiting. The chemical structures in the schemes herein depict variables that are hereby defined commensurately with chemical group definitions (moieties, atoms, etc.) of the corresponding position in the compound formulae herein, whether identified by the same variable name (i.e., R¹ or R²) or not. The suitability of a chemical group in a compound structure for use in the synthesis of another compound is within the knowledge of one of ordinary skill in the art. Additional methods of synthesizing compounds of Formula I and their synthetic precursors, including those within routes not explicitly shown in schemes herein, are within the means of chemists of ordinary skill in the art. Synthetic chemistry transformations and protecting group methodologies (protection and deprotection) useful in synthesizing the applicable compounds are known in the art and include, for example, those described in Larock R, Comprehensive Organic Transformations, VCH Publishers (1989); Greene T W et al., Protective Groups in Organic Synthesis, 3^{rd} Ed., John Wiley and Sons (1999); Fieser L et al., Fieser and Fieser's Reagents for Organic Synthesis, John Wiley and Sons (1994); and Paquette L, ed., Encyclopedia of Reagents for Organic Synthesis, John Wiley and Sons (1995) and subsequent editions thereof

Combinations of substituents and variables envisioned by this invention are only those that result in the formation of stable compounds.

### Pharmaceutical Compositions

Provided herein are compositions for use in treating agitation comprising a compound of Formula I (e.g., including any of the formulae herein), or a pharmaceutically acceptable salt of said compound; and an acceptable carrier. Also provided herein are compositions for use in treating a disease or disorder selected from the group consisting of diabetes, epilepsy, and depression, comprising a compound of Formula I (e.g., including any of the formulae herein), or a pharmaceutically acceptable salt of said compound; and an acceptable carrier. In one embodiment, the composition comprises an effective amount of the compound, or pharmaceutically acceptable salt thereof. In another embodiment, a composition of this invention further comprises a second therapeutic agent such as quinidine, or a pharmaceutically acceptable salt of quinidine. In one embodiment, the composition comprises an effective amount of the compound of Formula I, or pharmaceutically acceptable salt thereof, and an effective amount of quinidine or a pharmaceutically acceptable salt thereof. Preferably, a composition of this invention is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in pharmaceutical compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, 2006.

Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and United States patent publications 20060094744 and 20060079502.

The pharmaceutical compositions of the invention include those suitable for oral, rectal, nasal, topical (including buccal and sublingual), vaginal or parenteral (including subcutaneous, intramuscular, intravenous and intradermal) administration. In certain embodiments, the compound of the formulae herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, Md. (20th ed. 2000).

Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption.

In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are administered orally, the active ingredient is combined with emulsifying and suspending agents. If desired, certain sweetening and/or flavoring and/or coloring agents may be added.

Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of this invention may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g.: Rabinowitz J D and Zaffaroni A C, U.S. Pat. No. 6,803,031, assigned to Alexza Molecular Delivery Corporation.

Topical administration of the pharmaceutical compositions of this invention is especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compounds of this invention include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access.

Thus, according to yet another embodiment, the compounds of this invention may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to another embodiment, the invention provides a method of coating an implantable medical device comprising the step of contacting said device with the coating composition described above. It will be obvious to those skilled in the art that the coating of the device will occur prior to implantation into a mammal.

According to another embodiment, the invention provides a method of impregnating an implantable drug release device comprising the step of contacting said drug release device with a compound or composition of this invention. Implantable drug release devices include, but are not limited to, biodegradable polymer capsules or bullets, non-degradable, diffusible polymer capsules and biodegradable polymer wafers.

According to another embodiment, the invention provides an implantable medical device coated with a compound or a composition comprising a compound of this invention, such that said compound is therapeutically active.

According to another embodiment, the invention provides an implantable drug release device impregnated with or containing a compound or a composition comprising a compound of this invention, such that said compound is released from said device and is therapeutically active.

Where an organ or tissue is accessible because of removal from the subject, such organ or tissue may be bathed in a medium containing a composition of this invention, a composition of this invention may be painted onto the organ, or a composition of this invention may be applied in any other convenient way.

In one embodiment, a composition of this invention further comprises a second therapeutic agent wherein the second therapeutic agent is quinidine or quinidine sulfate.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In one embodiment of the pharmaceutical compositions of the invention, the compound of the present invention is present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, or enhance or improve the prophylactic or therapeutic effect(s) of another therapy.

The interrelationship of dosages for animals and humans (based on milligrams per meter squared of body surface) is described in Freireich et al., (1966) Cancer Chemother. Rep 50: 219. Body surface area may be approximately determined from height and weight of the subject. See, e.g., Scientific Tables, Geigy Pharmaceuticals, Ardsley, N.Y., 1970, 537.

In one embodiment, an effective amount of the compound of Formula I, or a pharmaceutically acceptable salt thereof, can range from about 1 mg to 1000 mg, from about 5 mg to 500 mg, from about 5 mg to 400 mg, from about 5 mg to 250 mg, from about 10 mg to 150 mg, from about 10 mg to 100 mg, or from about 5 mg to 50 mg, which can be given once, twice, or up to three times daily depending on various factors recognized by those skilled in the art. In one embodiment the effective amount of the compound of Formula I can be given once daily.

In one embodiment, the effective amount of quinidine, or a pharmaceutically acceptable salt thereof, can range from about 1 mg to 60 mg, from about 1 mg to 40 mg, from about 1 mg to 30 mg, from about 1 mg to 20 mg, from about 1 mg to 10 mg, or from about 1 mg to 5 mg, which can be given once, twice, or up to three times daily depending on various factors recognized by those skilled in the art. In one embodiment the effective amount of quinidine can be given once daily.

In certain embodiments, the amount of quinidine is an amount effective to increase the plasma half-life or decrease the intrinsic clearance of the compound of Formula I in the subject by at least 50%, 100%, 200%, 300%, 400%, 500%, 600%, 700%, 800%, 900%, or 1000%, relative to the plasma half-life or the intrinsic clearance of the compound of Formula I in the subject in the absence of quinidine.

In some embodiments, the method comprises administering an amount of the compound of Formula I in the range of 5 mg/day to 250 mg/day and the amount of quinidine is in the range of 1 mg/day to 20 mg/day.

In some embodiments, the method comprises administering an amount of the compound of Formula I in the range of 10 mg/day to 150 mg/day and the amount of quinidine is in the range of 1 mg/day to 20 mg/day.

Effective doses will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician. For example, guidance for selecting an effective dose can be determined by reference to the prescribing information for dextromethorphan or dimemorfan.

In some embodiments, a composition of this invention further comprises an additional therapeutic agent in an effective amount for the treatment of agitation, diabetes, epilepsy, or depression. In some embodiments, the agitation is associated with a disorder selected from the group consisting of Alzheimer's disease, a degenerative neurological disorder (e.g., Parkinson's disease, Huntington's disease, etc.), a mood disorder (e.g., depression, dysthymia, schizophrenia, bipolar disorder, etc.), substance abuse withdrawal, selective serotonin reuptake inhibitor (S SRI) withdrawal, withdrawal from benzodiazepines, withdrawal from drugs useful for the treatment of attention deficit disorder (ADD) and attention deficit hyperactive disorder (ADHD), traumatic brain injury, terminal illness, post-operative agitation, post-anesthetic agitation, Reye's syndrome and a pediatric disorder (e.g., depression, attention deficit disorder, oppositional defiant disorder, separation anxiety disorder, etc.).

For pharmaceutical compositions that comprise an additional therapeutic agent, an effective amount of the additional therapeutic agent is between about 0.01% to 100% of the dosage normally utilized in a monotherapy regime using just that agent. The normal monotherapeutic dosages of these additional therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the additional therapeutic agents referenced above will act synergistically with the compounds of this invention. When this occurs, it will allow the effective dosage of the additional therapeutic agent and/or the compound of this invention to be reduced from that required in a monotherapy. This has the advantage of minimizing toxic side effects of either the additional therapeutic agent or a compound of this invention, synergistic improvements in efficacy, improved ease of administration or use and/or reduced overall expense of compound preparation or formulation.

### Methods of Treatment

Provided herein are methods for treating agitation in a subject in need thereof, comprising administering a compound of Formula I, or a pharmaceutically acceptable salt thereof.

Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In some embodiments, the agitation is associated with a disorder selected from the group consisting of Alzheimer's disease, a degenerative neurological disorder, a mood disorder, substance abuse withdrawal, selective serotonin reuptake inhibitor (SSRI) withdrawal, withdrawal from benzodiazepines, withdrawal from drugs useful for the treatment of ADD and ADHD, traumatic brain injury, terminal illness, post-operative agitation, post-anesthetic agitation, Reye's syndrome and a pediatric disorder.

In some embodiments, the agitation is associated with a degenerative neurological disorder. In one aspect of these embodiments, the degenerative neurological disorder is Parkinson's disease. In another aspect of these embodiments, the degenerative neurological disorder is Huntington's disease.

In some embodiments, the agitation is associated with a mood disorder. In one aspect of these embodiments, the mood disorder is depression, dysthemia, schizophrenia or bipolar disorder. In one specific aspect of these embodiments, the mood disorder is depression. In one specific aspect of these embodiments, the mood disorder is dysthymia. In one specific aspect of these embodiments, the mood disorder is schizophrenia. In one specific aspect of these embodiments, the mood disorder is bipolar disorder.

In some embodiments, the agitation is associated with SSRI withdrawal. In one aspect of these embodiments, the SSRI is selected from fluoxetine, fluvoxamine, citalopram, escitalopram, paroxetine and sertraline.

In some embodiments, the agitation is associated with withdrawal from drugs useful for the treatment of ADD and ADHD. In one aspect of these embodiments, the drug useful for the treatment of ADD and ADHD is selected from methamphetamine hydrochloride, methylphenidate hydrochloride, dextroamphetamine sulfate, mixed amphetamine salts, pemoline, dexmethylphenidate hydrochloride, and lisdexamfetamine mesilate.

In some embodiments, the agitation is associated with a pediatric disorder. In one aspect of these embodiments, the pediatric disorder is depression, attention deficit disorder, oppositional defiant disorder, or separation anxiety disorder.

In some embodiments, the agitation is associated with Alzheimer's disease.

In some embodiments, the agitation is associated with traumatic brain injury.

In some embodiments, the invention provides a method of treating a subject suffering from agitation by co-administering to the subject in need thereof a compound of Formula I, or a composition comprising such compound; and quinidine, or a pharmaceutically acceptable salt of either or both thereof.

The term “co-administered” as used herein means that quinidine may be administered together with a compound of Formula I as part of a single dosage form (such as a composition of this invention comprising a compound of Formula and quinidine as described above) or as separate, multiple dosage forms. Alternatively, quinidine may be administered prior to, consecutively with, or following the administration of a compound of Formula I. In such combination therapy treatment, both the compound of Formula I and quinidine are administered by conventional methods. The administration of a composition of this invention, comprising both a compound of Formula I and quinidine, to a subject does not preclude the separate administration of a compound of Formula I, quinidine, or any other additional therapeutic agent to said subject at another time during a course of treatment.

In some embodiments, a method of this invention further comprises administering an additional therapeutic agent in an effective amount for the treatment of agitation, diabetes, epilepsy, or depression. In some embodiments, the agitation is associated with a disorder selected from the group consisting of Alzheimer's disease, a degenerative neurological disorder (e.g., Parkinson's disease, Huntington's disease, etc.), a mood disorder (e.g., depression, dysthymia, schizophrenia, bipolar disorder, etc.), substance abuse withdrawal, selective serotonin reuptake inhibitor (S SRI) withdrawal, withdrawal from benzodiazepines, withdrawal from drugs useful for the treatment of attention deficit disorder (ADD) and attention deficit hyperactive disorder (ADHD), traumatic brain injury, terminal illness, post-operative agitation, post-anesthetic agitation, Reye's syndrome and a pediatric disorder (e.g., depression, attention deficit disorder, oppositional defiant disorder, separation anxiety disorder, etc.).

Effective amounts of these additional therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the additional therapeutic agent's optimal effective-amount range. Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In yet another aspect, the invention provides the use of a compound of Formula I alone or together with quinidine, or a pharmaceutically acceptable salt of either or both thereof, in the manufacture of a medicament, either as a single composition or as separate dosage forms, for treatment or prevention in a subject of a disease, disorder or symptom set forth above. Another aspect of the invention is a compound of Formula I for use in the treatment or prevention in a subject of a disease, disorder or symptom thereof delineated herein.

Another aspect of the present invention is directed to methods for treating a disease or disorder selected from the group consisting of diabetes, epilepsy, and depression, comprising administering to a subject in need thereof an effective amount of a compound of Formula I, as described herein, or a pharmaceutically acceptable salt thereof, and quinidine or a pharmaceutically acceptable salt thereof (See Nature Medicine 21, 363-372 (2015) doi:10.1038/nm.3822; and WO 2013/029762) In some embodiments, treatment comprises the administration of a compound of Formula I, and quinidine, or a pharmaceutically acceptable salt of either or both thereof, and optionally a pharmaceutically acceptable carrier.

In some embodiments, the method comprises administering to the subject an amount of compound of Formula I, or a pharmaceutically acceptable salt thereof, in the range of 1 mg/day to 1000 mg/day, 5 mg/day to 500 mg/day, 5 mg/day to 400 mg/day, 5 mg/day to 250 mg/day, 10 mg/day to 150 mg/day, 10 mg/day to 100 mg/day, or 5 mg/day to 50 mg/day.

In some embodiments, the method comprises administering to the subject an amount of quinidine, or a pharmaceutically acceptable salt thereof, in the range of 1 mg/day to 60 mg/day, 1 mg/day to 40 mg/day, 1 mg/day to 30 mg/day, 1 mg/day to 20 mg/day, 1 mg/day to 10 mg/day, or 1 mg/day to 5 mg/day.

### EXAMPLES

Example 1. Synthesis of (+)-3-(Methyl-d₃)-17-methyl-(9α, 13α, 14α)-morphinan (102). Compound 102 was prepared as outlined below. Details of the synthesis are set forth below.

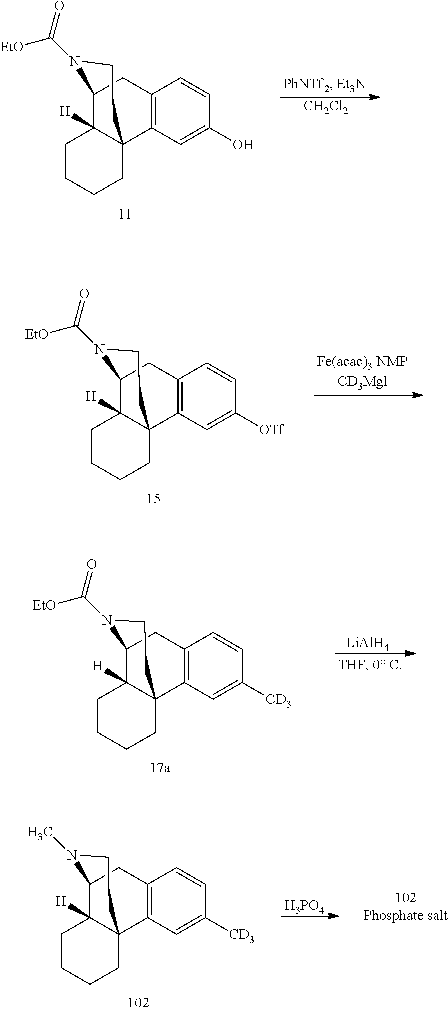

Synthesis of (+)-17-ethylcarbamate-3-trifluoromethylsulfonyloxy-(9α, 13α, 14α)-morphinan (15). To a solution of 11 (9 g, 28.6 mmol, prepared as in WO2010033801) and triethylamine (16 mL, 114 mmol) in CH2C12 (400 mL) was added N-phenyl-trifluoromethanesulfonimide “PhNTf₂” (20.7 g, 57.2 mmol) with cooling in an ice-bath. The reaction mixture was allowed to warm to ambient temperature and was stirred overnight. The mixture was diluted with CH₂Cl₂ (500 mL) and the solution was washed with saturated sodium bicarbonate, water, and brine, then dried over sodium sulfate. After filtration and concentration under reduced pressure, the crude product was purified by column chromatography on silica gel (ethyl acetate/heptanes, 0-10%) to afford 12 g (94%) of 15 as clear oil.

Synthesis of (+)-17-ethylcarbamate-3-(methyl-d₃)-(9α, 13α, 14α)-morphinan (17a). To a solution of 15 (22 g, 43.8mmol ) in THF (500 mL) was added N-methyl-2-pyrrolidone “NMP” (26.2 mL, 153.1 mmol) at ambient temperature. The reaction mixture was degassed by N₂ purge for 10 minutes. Iron(III) acetylacetonate “Fe(acac)₃” (1.65 g, 4.4 mmol) and CD₃MgI (1 M in Et₂O, 53 mL, 47.6 mmol, Sigma Aldrich, 99 atom% D) were added and the reaction mixture was heated to reflux overnight. The reaction was cooled and water (500 mL) was added. The layers were separated and the aqueous layer was extracted with CH₂Cl₂ (3×100 mL). The combined organics were washed with brine, dried over sodium sulfate, filtered, concentrated under reduced pressure and purified by column chromatography on silica gel (ethyl acetate/heptanes, 0-10%) to afford 4 g (94%, based on recovered starting material) of 17 a and 16 g of recovered 15.

Synthesis of (+)-3-(methyl-d₃)-17-methyl-(9α, 13α, 14α)-morphinan (102). A mixture of 17 a (1.5 g, 4.8 mmol) in THF (70 mL) was treated with LiAlH₄ (1 M in THF, 19.2 mL) at 0 ° C. The mixture was allowed to warm to ambient temperature and was stirred overnight. Water (1 mL) was added to quench the reaction, followed by NaOH (24%, 10 mL). The mixture was stirred for 30 minutes, during which time white solid precipitated. The solid was filtered and the filtrate was concentrated under reduced pressure. The crude product was purified by preparative HPLC (see conditions described below) to afford 102. The free amine was dissolved in MTBE (30 mL) and heated to reflux. H₃PO₄ (in isopropanol) was added dropwise, resulting in the formation of a white solid. The addition of H3PO4 was continued until no more white solid appeared to precipitate. The solid was filtered and washed with MTBE (100 mL) to provide 1.2 g of solid. The material was recrystallized with MeOH/MTBE to provide 102 as the phosphate salt (0.75 g, 47%).

¹H-NMR (300 MHz, CD₃OD): δ1.07-1.57 (m, 8H), 1.69-1.72 (m, 1H), 1.96-2.10 (m, 2H), 2.56 (br.d., 1H), 2.91 (s, 3H), 3.06-3.11 (br. s. and m, 3H), 3.56 (br.m., 1H), 7.03-7.18 (m, 3H). HPLC (method: 20 mm C-18 RP column - gradient method 2-95% ACN/water/0.1% formic acid; Wavelength: 210 nm): retention time: 2.59 min, purity: 99.4%. MS (M+H): 259.2

### Preparative HPLC Conditions

Sunfire C18 5 μm 30×150mm column; Waters GI Pump;
Solvent A=water; Solvent B=acetonitrile

| Time (min) | Flow Rate (mL/min) | % A | % B |
|---|---|---|---|
| 0 | 40.00 | 90 | 10 |
| 7.00 | 40.00 | 50 | 50 |
| 8.00 | 40.00 | 5 | 95 |
| 9.00 | 20.00 | 90 | 10 |
| 10.00 | 20.00 | 90 | 10 |

Example 2. Synthesis of (+)-3-Methyl-17-(methyl-d₃)-(9α, 13α, 14α)-morphinan (101). Compound 101 was prepared as outlined below. Details of the synthesis follow.

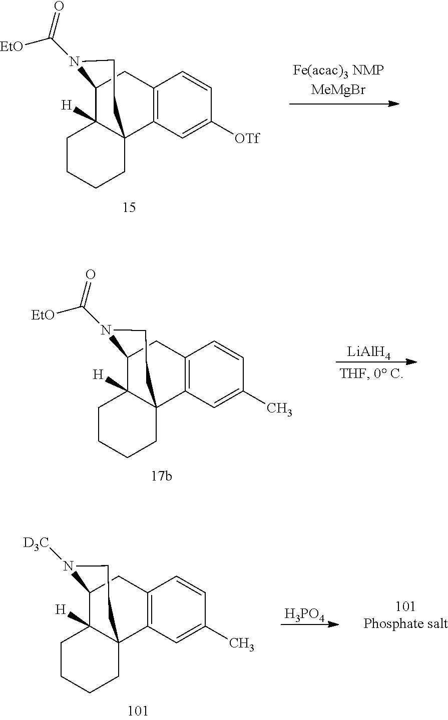

Synthesis of (+)-17-ethylcarbamate-3-methyl-(9α, 13α, 14α)-morphinan (17b). To a solution of 15 (3.6 g, 7.16 mmol, see Example 1) in THF (100 mL) was added N-methyl-2-pyrrolidone “NMP” (4.3 mL, 25.1 mmol) at ambient temperature. The reaction mixture was degassed by N₂ purge for 10 minutes. Iron(III) acetylacetonate “Fe(acac)₃” (270 mg, 0.72 mmol) and MeMgBr (3M in Et₂O, 2.9 mL, 7.8 mmol) were added and the reaction mixture was heated to reflux overnight. The reaction was cooled and water (50 mL) was added. The layers were separated and the aqueous layer was extracted with CH₂Cl₂ (3×100 mL). The combined organics were washed with brine, dried over sodium sulfate, filtered, concentrated under reduced pressure, and purified by column chromatography on silica gel (ethyl acetate/heptanes, 0-10%) to give 0.84 g of 17b (75% based on recovered starting material) and 2 g of recovered 15.

Synthesis of (+)-3-methyl-17-(methyl-d₃)-(9α, 13α, 14α)-morphinan (101). A mixture of 17b (1 g, 6.2 mmol) in THF (30 mL) was treated with LiAlD₄ (0.9 g, 24.8 mmol, Cambridge Isotopes, 98 atom% D) at 0° C. and the reaction was allowed to warm to ambient temperature and stir overnight. Water (1 mL) was added to quench the reaction, followed by NaOH (24%, 5 mL). The mixture was stirred for 30 minutes, during which time white solid precipitated. The solid was filtered and the filtrate was concentrated under reduced pressure. The crude product was dissolved in EtOAc (30 mL) and extracted with 10% HCl (3×30 mL). The combined aqueous layer was washed with CH₂Cl₂ (30 mL) and neutralized with 10% NaOH. The aqueous layer was then extracted with CH2Cl₂ (3×30 mL), the combined organics were dried over sodium sulfate, filtered and concentrated under reduced pressure to afford 101. The free amine was dissolved in MTBE (30 mL) and heated to reflux. H₃PO₄ (in isopropanol) was added dropwise, resulting in the formation of a white solid. The addition of H₃PO₄ was continued until no more white solid appeared to precipitate. The solid was filtered and washed with MTBE (100 mL). The product was recrystallized with MeOH/MTBE to provide 101 as the phosphate salt (0.4 g, 36%).

¹H-NMR (300 MHz, CD₃OD): δ1.09-1.60 (m, 7H), 1.68-1.71 (m, 1H), 1.98-2.02 (m, 1H), 2.04-2.15 (m, 1H), 2.31 (s, 3H), 2.50-2.55 (m, 1H), 2.64-2.65 (m, 1H), 3.06-3.07 (m, 1H), 3.16 (br.s., 2H), 3.54-3.55 (m, 1H), 7.02-7.17 (m, 3H). ¹³C-NMR (75 MHz, CD₃OD): δ20.2, 21.7, 25.8, 35.0, 35.8, 60.3, 125.8, 127.4, 128.0, 130.8, 137.2, 137.4. HPLC (method: 20 mm C18 RP column-gradient method 2-95% ACN/water/0.1% formic acid; Wavelength: 210 nm):-retention time: 2.51 min. purity: 97.7%. MS (M+H): 259.2.

Example 3. Synthesis of (+)-3-(Methyl-d3)-17-(methyl-d3)-(9α, 13α, 14α)-morphinan (100). Compound 100 was prepared as outlined below. Details of the synthesis follow.

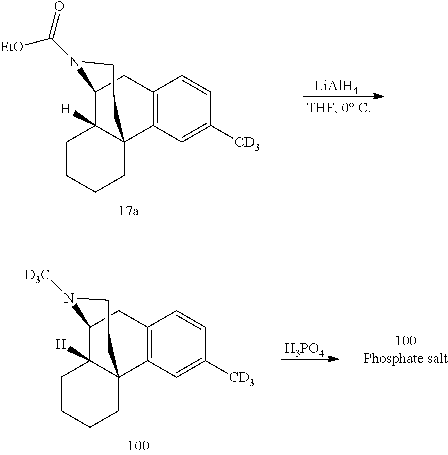

Synthesis of (+)-3-(methyl-d₃)-17-(methyl-d₃)-(9α, 13α, 14α)-morphinan (100). A mixture of 17a (2.5 g, 8 mmol, see Example 1) in THF (70 mL) was treated with LiAlD₄ (1.7 g, 32 mmol, Cambridge Isotopes, 98 atom% D) at 0° C. The mixture was allowed to warm to ambient temperature and was stirred overnight. Water (1 mL) was added to quench the reaction, followed by NaOH (24%, 10 mL). The mixture was stirred for 30 minutes, during which time white solid precipitated. The solid was filtered and the filtrate was concentrated under reduced pressure. The crude product was purified by preparative HPLC (see conditions described in Example 1) to provide 100. The free amine was dissolved in MTBE (50 mL) and was heated to reflux. H₃PO₄ (in isopropanol) was added dropwise, resulting in the formation of a white solid. The addition of H₃PO₄ was continued until no more white solid appeared to precipitate. The solid was filtered and washed with MTBE (100 mL) to provide 1.2 g of solid. The product was recrystallized in MeOH/MTBE to provide 100 as the phosphate salt (1 g, 36%).

¹H-NMR (300 MHz, CD₃OD): δ1.09-1.13 (m, 1H), 1.24-1.33 (m, 1H), 1.39-1.72 (m, 6H), 1.95-2.04 (m, 1H), 2.16-2.18 (m, 1H), 2.50-2.54 (m, 1H), 2.60-2.68 (m, 1H), 3.07-3.16 (m. and s., 3H), 3.54-3.55 (m, 1H), 7.02-7.17 (m, 3H). ¹³C-NMR (75 MHz, D₂O): δ21.4, 23.2, 25.5, 25.6, 34.7, 39.3, 43.0, 47.8, 60.4, 126.4, 127.5, 128.3, 131.2, 138.0. HPLC (method: 20 mm C18 RP column-gradient method 2-95% ACN/water/0.1% formic acid; Wavelength: 210 nm): retention time: 2.61 min., purity >99.9%. MS (M+H): 262.2.

Example 4. Determination of Metabolic Stability of Test Compounds using Human Liver Microsomes. Human liver microsomes (20 mg/mL) were obtained from Xenotech, LLC (Lenexa, Kans.). β-nicotinamide adenine dinucleotide phosphate, reduced form (NADPH), magnesium chloride (MgCl₂), and dimethyl sulfoxide (DMSO) were purchased from Sigma-Aldrich.

7.5 mM stock solutions of test compounds were prepared in DMSO. The 7.5 mM stock solutions were diluted to 50 μM in acetonitrile (ACN). The 20 mg/mL human liver microsomes were diluted to 1.25 mg/mL (1 mg/mL final) in 0.1 M potassium phosphate buffer, pH 7.4, containing 3 mM MgCl₂. The diluted microsomes (375 μL) were added to wells of a 96-well polypropylene plate in triplicate. 10 μL of the 50 μM test compound was added to the microsomes and the mixture was pre-warmed for 10 minutes. Reactions were initiated by addition of 125 μL of pre-warmed NADPH solution. The final reaction volume was 0.5 mL and contained 1.0 mg/mL human liver microsomes, 1 μM test compound, and 2 mM NADPH in 0.1 M potassium phosphate buffer, pH 7.4, and 3 mM MgCl₂. The reaction mixtures were incubated at 37° C., and 50 μL aliquots were removed at 0, 5, 10, 20, and 30 minutes and added to shallow 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems API 4000 mass spectrometer.

7-ethoxy coumarin was used as a positive control.

The in vitro t_{1/2}s for test compounds were calculated from the slopes of the linear regression of % parent remaining (In) vs incubation time relationship:

in vitro t _{1/2}=0.693/k, where k=-[slope of linear regression of % parent remaining(In) vs incubation time]

Data analysis was performed using Microsoft Excel Software.

Table 2 shows the results of this experiment.

TABLE 2
Calculated Half-life in Human Liver Microsomes

| | | Change over |
|---|---|---|
| | Ave. t_{1/2} | non-deuterated |
| Compound | (n = 2) | compound |
| Dimemorfan | 23.1 | n/a |
| Compound 102 | 28.0 | 21% |
| Compound 100 | 31.6 | 37% |

In the case of dimemorfan, deuteration of R¹ resulted in a significant increase in half life (t_{1/2}) in human liver microsomes as compared to the undeuterated counterpart. Deuteration of the N-methyl moiety (R²) caused a further significant increase in t_{1/2}.

Example 5. Effect of Quinidine on the Metabolic Stability of Dextromethorphan, Dimemorfan, and Deuterated Dimemorfan in Human Liver Microsomes

### Materials and Methods

Materials: Individual donor with extensive metabolizer human liver microsomes (EM HLM, 20 mg/mL) with CYP2D6 activity of 957±19 pmol/mg protein/min, genotyped for CYP2D6 allelic variant-CYP2D6*1/*2x2, were obtained from Xenotech, LLC (Lenexa, Kans.). β-nicotinamide adenine dinucleotide phosphate, reduced form (NADPH), magnesium chloride (MgCl₂), dimethyl sulfoxide (DMSO), acetonitrile (ACN), quinidine, and dextromethorphan (DM) were purchased from Sigma-Aldrich. Dimemorfan and d₆-dimemorfan (Compound 100) were synthesized by Adesis, Inc.

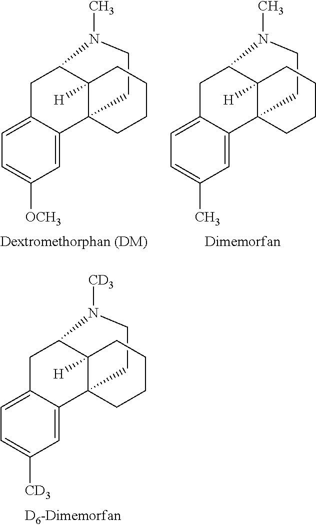

Determination of Metabolic Stability: 1.0 mg/mL stock solutions of test compounds dextromethorphan, dimemorfan, and d6-dimemorfan (Compound 100) were prepared in DMSO. The 1.0 mg/mL stock solutions were diluted to 10,000 ng/mL in acetonitrile (ACN). A 10 mM stock solution of quinidine was prepared in acetonitrile, which was then diluted further in acetonitrile to produce 20 μM, 10 μM, 5 μM, 2.5 μM, and 1.25 μM solutions of quinidine. The human liver microsomes were diluted to 1.25 mg/mL in 0.1 M potassium phosphate buffer, pH 7.4, containing 3 mM MgC12. The diluted microsomes were added to wells of a 96-well deep-well polypropylene plate in triplicate. A 5 μL aliquot of each 10,000 ng/mL test compound was added to the microsomes, and the mixture was pre-warmed at 37° C. for 7 minutes, then a 5 μL aliquot of each quinidine solution was added (except in control wells where no quinidine was added). Reactions were initiated by addition of pre-warmed NADPH solution to the mixture. The final reaction volume was 0.5 mL and contained 1.0 mg/mL human liver microsomes, 100 ng/mL of test compound, 0.2, 0.1, 0.05, 0.025, 0.0125, or 0 μM of quinidine, and 2 mM NADPH in 0.1 M potassium phosphate buffer, pH 7.4, and 3 mM MgCl₂. The reaction mixtures were incubated at 37° C., and 50 μL aliquots were removed at 0, 15, 30, and 60 minutes and added to shallow-well 96-well plates which contained 50 μL of ice-cold ACN with internal standard to stop the reactions. The plates were stored at 4° C. for 20 minutes after which 100 μL of water was added to the wells of the plate before centrifugation to pellet precipitated proteins. Supernatants were transferred to another 96-well plate and analyzed for amounts of parent remaining by LC-MS/MS using an Applied Bio-systems mass spectrometer. Amount of parent remaining was quantified via standard curve.

### Data Analysis

The in vitro t_{1/2}'s for test compounds were calculated from the slopes of the linear regression of % parent remaining (In) vs. incubation time relationship.
in vitro t_{1/2}=0.693/k
k=-[slope of linear regression of % parent remaining(In) vs incubation time]

The apparent intrinsic clearance was calculated using the following equation:

CLᵢₙₜ (mL/min/kg)=(0.693/in vitro t_{1/2}) (Incubation Volume/mg of microsomes)

(45 mg microsomes/gram of liver) (20 gm of liver/kg b.w.)

Data analysis was performed using Microsoft Excel Software. Tables 3-6 show the results of these experiments.

TABLE 3
Calculated Half-life Ratio in Extensive
Metabolizer Human Liver Microsomes

| | t_{1/2} (min) | | |
|---|---|---|---|
| Quinidine | t_{1/2} Ratio (respective to 0 concentration of Quinidine) | | |
| Conc (μM) | DM | Dimemorfan | D6-Dimemorfan |
| 0.2 | 64.5* | 43.1 | 37.9 |
| | 3.1 | 9.0 | 5.8 |
| 0.1 | 42.1 | 25.5 | 31.1 |
| | 2.1 | 5.3 | 4.8 |
| 0.05 | 33.0 | 16.2 | 18.0 |
| | 1.6 | 3.4 | 2.8 |
| 0.025 | 29.6 | 12.8 | 13.2 |
| | 1.4 | 2.7 | 2.0 |
| 0.0125 | 25.6 | 6.34 | 10.1 |
| | 1.2 | 1.3 | 1.6 |
| 0 | 20.5 | 4.81 | 6.5 |
| | 1.0 | 1.0 | 1.0 |

*t_{1/2} exceeds the time of the experiment
EM HLM, 1.0 mg/mL, 100 ng/mL, 60 min
*Bottom number in each cell is the t_{1/2} ratio respective to t_{1/2} at a concentration of 0 μM quinidine.

TABLE 4
Extensive Metabolizer Human Liver Microsomes:
Deuterium Effect on Half-life

| | | | | % Δ |
|---|---|---|---|---|
| Quinidine | t_{1/2} (min) | | | (D6-Dimemorfan/ |
| Conc (μM) | DM | Dimemorfan | D6-Dimemorfan | Dimemorfan) |
| 0.2 | 64.5* | 43.1 | 37.9 | −12% |
| 0.1 | 42.1 | 25.5 | 31.1 | 22% |
| 0.05 | 33.0 | 16.2 | 18.0 | 11% |
| 0.025 | 29.6 | 12.8 | 13.2 | 3% |
| 0.0125 | 25.6 | 6.34 | 10.1 | 59 |
| 0 | 20.5 | 4.81 | 6.5 | 35 |

*t_{1/2} exceeds the time of the experiment
EM HLM, 1.0 mg/mL, 100 ng/mL, 60 min

TABLE 5
Calculated Intrinsic Clearance in Extensive
Metabolizer Human Liver Microsomes

| | Cl_{int, app} (mL/min/kg) | | |
|---|---|---|---|
| Quinidine | Cl_{int, app} Ratio (respective to 0 concentration of Quinidine) | | |
| Conc (μM) | DM | Dimemorfan | D6-Dimemorfan |
| 0.2 | 4.8 | 7.4 | 8.2 |
| | 0.31 | 0.11 | 0.17 |
| 0.1 | 7.4 | 12.2 | 10.0 |
| | 0.48 | 0.19 | 0.21 |
| 0.05 | 9.5 | 19.3 | 17.3 |
| | 0.62 | 0.30 | 0.36 |
| 0.025 | 10.6 | 24.7 | 23.6 |
| | 0.69 | 0.38 | 0.49 |
| 0.0125 | 12.1 | 50.8 | 30.9 |
| | 0.79 | 0.78 | 0.64 |
| 0 | 15.4 | 64.9 | 48.2 |
| | 1.0 | 1.0 | 1.0 |

CLᵢₙₜ = (0.693/in vitro t_{1/2}) (Incubation Volume/mg of microsomes) (45 mg microsomes/gram of liver) (20 gm of liver/kg b.w.)
* Bottom number in each cell is the intrinsic clearance ratio respective to the intrinsic clearance at a concentration of 0 μM quinidine.

TABLE 6
Extensive Metabolizer Human Liver Microsomes:
Deuterium Effect on Intrinsic Clearance

| | | | | Ratio |
|---|---|---|---|---|
| Quinidine | Cl_{int, app} (mL/min/kg) | | | (D6-Dimemorfan/ |
| Conc (μM) | DM | Dimemorfan | D6-Dimemorfan | Dimemorfan) |
| 0.2 | 4.8 | 7.4 | 8.2 | 1.11 |
| 0.1 | 7.4 | 12.2 | 10.0 | 0.82 |
| 0.05 | 9.5 | 19.3 | 17.3 | 0.90 |
| 0.025 | 10.6 | 24.7 | 23.6 | 0.96 |
| 0.0125 | 12.1 | 50.8 | 30.9 | 0.61 |
| 0 | 15.4 | 64.9 | 48.2 | 0.74 |

CLᵢₙₜ = (0.693/in vitro t_{1/2}) (Incubation Volume/mg of microsomes) (45 mg microsomes/gram of liver) (20 gm of liver/kg b.w.)

In the above Table 4, if the apparent intrinsic clearance ratio (D₆-dimemorfan/dimemorfan) is >1.15 or <0.85, then there is considered to be differentiation between D₆-dimemorfan and dimemorfan.

The results show that quinidine increases the t_{1/2} ratio (and decreases the intrinsic clearance ratio) of dimemorfan in this assay more than quinidine increases the t_{1/2} ratio (and decreases the intrinsic clearance ration) of dextromethorphan (DM) in this assay, for a given concentration of quinidine.

Without further description, it is believed that one of ordinary skill in the art can, using the preceding description and the illustrative examples, make and utilize the compounds of the present invention and practice the claimed methods. It should be understood that the foregoing discussion and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

1-14. (canceled).

15. A method of treating a disease or disorder selected from the group consisting of diabetes, epilepsy, and depression, comprising administering to a subject in need thereof an effective amount of a compound of Formula I: 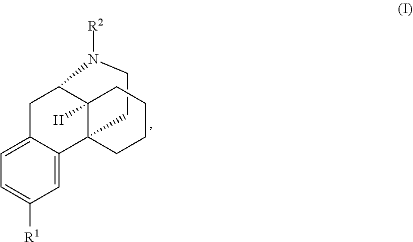
or a pharmaceutically acceptable salt thereof, wherein:
R¹ is selected from —CH₃, —CH₂D, —CHD₂, and —CD₃; and
R² is selected from CH₃, CH₂D, CHD₂, and CD₃;
provided that either R¹ or R² comprises at least one deuterium atom;
and quinidine or a pharmaceutically acceptable salt thereof; and a pharmaceutically acceptable carrier.

16. The method of claim 15, wherein R¹ is CH₃ or CD₃ and R² is CH₃ or CD₃.

17. The method of claim 15, wherein for the compound of Formula I, any atom not designated as deuterium is present at its natural isotopic abundance.

18. The method of claim 17, wherein the compound of Formula I is a compound selected from the table:

| Compound No. | R¹ | R² |
|---|---|---|
| 100 | —CD₃ | —CD₃ |
| 101 | —CH₃ | —CD₃ |
| 102 | —CD₃ | —CH₃, |

or a pharmaceutically acceptable salt thereof.

19. The method of claim 15, wherein the amount of the compound of Formula I, or a pharmaceutically acceptable salt thereof, is in the range of 5 mg/day to 500 mg/day, and the amount of quinidine, or a pharmaceutically acceptable salt thereof, is in the range of 1 mg/day to 40 mg/day

20. The method of claim 15, wherein the amount of the compound of Formula I, or a pharmaceutically acceptable salt thereof, is in the range of 5 mg/day to 250 mg/day and the amount of quinidine, or a pharmaceutically acceptable salt thereof, is in the range of 1 mg/day to 20 mg/day.

21. The method of claim 15, wherein the amount of the compound of Formula I, or a pharmaceutically acceptable salt thereof, is in the range of 10 mg/day to 150 mg/day and the amount of quinidine, or a pharmaceutically acceptable salt thereof, is in the range of 1 mg/day to 20 mg/day.

22-23. (canceled).

24. The method of claim 15, wherein the deuterium incorporation at each designated deuterium atom is at least 90%.

25. The method of claim 15, wherein the deuterium incorporation at each designated deuterium atom is at least 95%.
